(19) United States
(12) Patent Application Publication
(10) Pub. No.: US 2006/0058737 A1
(43) Pub. Date: Mar. 16, 2006

(54) Catheter treatment stylet

(76) Inventors: Steve A. Herweck, Nashua, NH (US); Paul Martakos, Pelham, NH (US); Geoffrey Moodie, Hudson, NH (US); Joseph Ferraro, Londonderry, NH (US)

Correspondence Address: LAHIVE & COCKFIELD, LLP., 28 STATE STREET, BOSTON, MA, 02109, US

(21) Appl. No.: 10/944,393

(22) Filed: Sep. 16, 2004

(51) Int. Cl. A61M 5/178

(52) U.S. Cl. 604/164.01

(57) ABSTRACT

A therapeutic agent-eluting stylet treats a catheter by at least temporarily locating the stylet in a lumen of an indwelling catheter placed into a patient. The therapeutic agent-carrying stylet provides a localized therapeutic effect to the catheter and patient by eluting one or more therapeutic agents directly into the lumen of the catheter, into the fluid within the catheter, into the fluid and tissue in contact with the catheter, and onto the surface of the catheter. By removal and replacement of the temporary stylet from the catheter, renewable therapeutic agent doses can be provided to a component, such as the lumen, of the catheter while the catheter remains within in a patient. One or more therapeutically effective doses of medicated agents can be tailored along the length of the stylet and controlled by the length of time by which the stylet remains installed in the catheter.

### FIELD OF THE INVENTION

The present invention relates to treatment of a catheter. More specifically, the invention relates to an insertable and removable device for applying a therapeutic agent to a selected portion of a catheter for applications, such as minimizing undesired growth within a catheter, thereby limiting blockage of holes at or near the end of the catheter and/or reducing a likelihood of infection.

### BACKGROUND OF THE INVENTION

A number of patients today undergo recurring medical procedures requiring repeated skin penetrating access to the patient's vascular system for life sustaining medical procedures using a catheter. A catheter is generally installed in a body fluid-containing organ, such as the circulatory vascular system, endocrine system and urological organs via a communicating catheter lumen passageway. The catheter establishes a channel or passageway to other internal tissue locations requiring access, such as in the mediastinal, chest, abdominal and peritoneal cavities, as well as spinal spaces, bladder, duct or cyst type cavities.

One such recurring medical procedure requiring repeat catheter access or passageway to the patient's circulatory system is hemodialysis. Currently, over one million patients worldwide suffer from End State Renal Disease (ESRD) conditions and require some form of daily or thrice weekly dialysis treatment via needle or catheter access. Peritoneal Dialysis is one form of dialysis treatment requiring needle or catheter access whereby fluids placed into the peritoneal cavity via a temporary or permanently placed access catheter provide osmotic transfer of blood containing toxins into solutions pumped into and removed from within the peritoneal organ cavity. A second form of dialysis treatment is a direct blood filtering process, whereby a needle or catheter is placed directly into a vein or artery, and through a series of connecting tubing, blood is removed and re-circulated back into the patient after filtration of the blood. These two hemodialysis procedures are the most common means for metabolic toxin removal from body fluids when a patient experiences total or bilateral renal failure.

One of the more popular dialysis access methods is a long term catheter option. Unlike the other vascular access options, which are accessed by a needle through the skin, an indwelling dialysis catheter provides a needleless connection system. Surgical implantation is often done by interventional radiology means under fluoroscopy and can be used for dialysis immediately after installation. Currently available commercial dialysis catheters usually have two external tubes that extend from the patient's upper chest or neck to allow for a dialysis nurse or technician to connect the patient directly to the dialysis machine via the tubing. Maintenance of the catheters is performed through the tubes, and requires constant hygiene care to avoid internal occlusion and infection. Any infection that develops in or near the catheters requires immediate device removal and systemic antibiotic medication administration, due to the location of the distal tips of the catheters in or near the heart via the main vena cava blood vessel. Therefore, infection of the catheters can be a devastating and life-threatening event, as the patient requires dialysis treatment to avoid extreme levels of blood toxicity, and emergency hospitalization for dialysis treatment must be implemented at great cost.

Peritoneal dialysis, for example, generally requires surgical implantation of a long term or extended implant period dialysis access catheter. Peritoneal dialysis access (PDA) catheters are surgically installed thru a small skin incision, with an extended length of catheter material placed in and around the intestinal organs within the peritoneal abdominal cavity. During peritoneal dialysis treatment, dialysis fluids are placed temporarily into this closed internal body cavity via the peritoneal dialysis access catheter. This non-vascular, abdominal dialysis catheter method allows for large volumes of fluid to be cycled into and out of the closed internal body cavity, exclusively for the liquid osmotic transfer of blood toxins out of the patient's blood. The indwelling catheter forces the blood toxins, which circulate near the surface of the abdominal wall cavity lining, into the temporary placed fluid solutions entered into the cavity by the indwelling catheter. Toxin removal out of the patient occurs by natural liquid osmotic transfer or chemical exchange into the abdominal fluids cycled through the abdominal cavity by such implantable catheters. Typically, most peritoneal dialysis fluids are pumped into and removed from within this closed abdominal cavity via the surgically installed catheter, catheter connection means and sterile connecting tubing, which are connected in series with a dialysis fluid container and programmable peritoneal dialysis pump device.

It is widely known that foreign material formed in diagnostic or therapeutic catheters that penetrate through and are left to protrude through the patients protective skin for long periods of time, such indwelling catheters, can easily become infected or contaminated by naturally occurring growths, such as biofilms and thrombus forming proteins without aggressive therapeutic and preventative infection control measures. To maintain full functionality of any indwelling catheter while implanted, it is desirable to minimize naturally occurring biological blockage of the catheter lumen, and to prevent any catheter openings from becoming permanently occluded while the catheter remains implanted for extended periods of time.

Conventional infection and thrombus occlusion prevention involves application of a liquid catheter lock solution, containing some form of therapeutic agent, to help prevent infection or thrombus occlusion of the indwelling catheter between dialysis treatments. Therapeutic liquid catheter lock solutions are often applied to the indwelling catheter lumen after the conclusion of every dialysis treatment, and are sometimes repeatedly performed after each dialysis treatment for the life of the catheter's useful life to help prevent occlusion and or infection complications. Liquid catheter lock solutions must be carefully measured and administered by a healthcare worker to ensure that no more than the maximum luminal volume capacity of the implanted catheter device is injected, and whose volume must be keep within the device by sealing and closing off the connection end of the catheter via a cap or leak proof connection means. Sealing or capping off one end of the catheter, with the liquid lock solution installed within, relies on a mechanical vacuum force to retain the liquid within the closed surface container, whereby the three closed sides of the indwelling catheter lumen maintain a sufficient vacuum so as to keep a majority of the liquid lock solution inside the lumen of the catheter. Such carefully measured and administered therapeutic liquid solutions are applied to the catheter at the end of each dialysis treatment, and subsequently removed from the implanted catheter device and discarded, prior to the start of another dialysis treatment.

Typically, liquid catheter lock solutions intended for hemodialysis catheter use are formed of measured volumes having higher than systemically recommended concentrations of anti-coagulant medications, such a Heparin or tissue plasminogen activators like TPA, and are used in combination with other therapeutic agents which exhibit antiseptic or anti-infective effects at high, non-systemic concentrations. If the liquid catheter lock solution, having the higher than normally recommended systemic level concentrations, were accidentally injected out of the catheter and into the patient's venous and pulmonary circulatory system, serious side effects, including death, can potentially result. Therefore, only professionally trained healthcare workers are allowed to administer such liquid catheter lock solutions, including medication dosing preparation, sterile solution delivery preparation, insertion and removal from the catheter. The dialysis healthcare worker must be careful to minimize unavoidable wicking of small portions of the liquid catheter lock solution, which can occur at, along or near any catheter openings of the catheter, as most liquid catheter lock solutions are not intended for systemic use due to the high risk of complications which can result from the high concentrations of antiseptic or therapeutic agents required to avoid indwelling patient catheter complications.

Very often, an anticoagulant such as Heparin or CDA Citrate solution is used in combination with other therapeutic agents in the liquid catheter lock solution. The anticoagulant is injected into the indwelling catheter lumen to help minimize thrombotic occlusion of the catheter during the period that the patient is not undergoing hemodialysis treatment or when the indwelling catheter remains not in use. However, such liquid catheter lock solutions have been reported to not to achieve the desired therapeutic effects, due to the risks of systemic complications of such therapeutic agents, and the high concentrations of medication required to achieve an effective localized effect within the catheter. As an example, liquid solutions containing high concentrations of Heparin are now known to harbor and encourage bacterial colonization, principally due to Heparin's polysaccharide chemical composition which promotes bacterial growth. Therefore, in an effort to prevent thrombotic occlusion with a liquid catheter lock solution containing Heparin, the healthcare worker can predispose the dialysis catheter and patient to a higher incidence to bacterial infection. Bacterial infection can be a worse complication than thrombotic occlusion, as catheter infection renders the catheter non-functional during the period in which it is infected, and unfortunately many such catheter related infections require the removal of this dialysis treatment access device from the patient, a devastating complication for most patients.

If bacterial colonization infection and/or occlusive thrombus or occlusive protein formation can not be remedied by these liquid solution procedures, the entire catheter must be removed. The patient must then be placed on an aggressive systemic antibiotic medication program until the risk of infection has completely resolved, and/or the risk of infection remains in control, and the patient's elevated body temperature has returned to near normal levels. Only after the patient's elevated body temperature returns to normal, the condition of the local infected tissue and catheter site has returned to near normal conditions and the patient's blood chemistry has been determined to be adequately improved, would another new dialysis catheter device be installed. These types of catheter related complications and current preventative maintenance therapies can be a significant problem for many dialysis patients who cannot tolerate even temporary loss of use of their dialysis access connections means to survive for extended periods of time without adequate hemodialysis treatment.

When liquid anti-thrombotic or anti-clot agents such as CPDA solution are used as a catheter lock solution for hemodialysis catheters, such agents can be harmful to the patient if accidental systemic release were to occur. Citrate containing solutions commonly used to keep blood cells from sticking to each other in blood storage bags, are frequently used as a low cost alternative anticoagulant catheter lock solutions for hemodialysis catheters. While citrate may not promote infection as readily as heparin, systemic exposure of citrate to the heart, caused, for example, by accidental entry of a citrate solution the patient's systemic circulation, has been reported to cause significant patient complications, including pulmonary occlusion and cardiac arrest.

Other preventive therapeutic measures used by healthcare workers and patients to help avoid catheter related dialysis catheter infection include use of topically applied anti-infective medications. The use of topically applied anti-septic or anti-infective lotions, ointments or gel like films only treat the exposed surfaces of such catheter surfaces, and thus have little or no therapeutic effect to the indwelling portion of the catheter or catheter lumen, which are the location of the patient's catheter device related occlusion and or infection complications.

Another approach that has been reported in the art to help avoid such catheter related complications is to coat such implantable catheters with special biocompatible polymers and therapeutic agents in the form of outside and inside surface coatings. Unfortunately, most active medications lose their activity within hours of exposure to body fluids and contact with local tissue. Some medicated coatings have been shown not to work for longer than the first 48 hours, and frequently such coatings have been documented to incur various levels of inflammation after the medication has leached out from the drug carrier coating. It has also been documented that with the formation of the bacterial biofilms and other biological or protein sourced growths that form around such indwelling catheters, such biological activity tends to wall-off the medicated coatings from the surrounding tissue, reducing or ending their initial clinical effectiveness, even after only 48 hours in the body. Therefore, such therapeutic catheter coatings do not provide a long term therapeutic function, or extended therapeutic effect shortly after catheter placement within the body. Most therapeutic or medicated coatings stop working after a short period of time due to the short half life of the medication applied to the coating, and therefore such medicated catheter coatings do not provide effective long term therapeutic relief from catheter related complications commonly experienced by most patients. It is also well understood that many polymeric drug delivery coatings used as therapeutic agent delivery coatings on medical devices exhibit some form of chronic inflammatory reactions to blood and local tissue, during and after the therapeutic agent has migrated out from the carrier coating, especially when such devices are left in the body for extended periods of time. Therefore, many coated catheter and intervention devices coated with even the most effective antibiotics and antiseptic agents, such as rifampin and minocycline, fail to achieve a long-term treatment effect for the prevention of biofilm formation, localized tissue infection in contact with such devices, proliferation of invaginating tissue and/or prevent chronic inflammation and thrombotic occlusion. The potential use of these coated catheter and intervention devices on many medical devices such as hemodialysis access catheters is therefore limited.

### SUMMARY OF THE INVENTION

A therapeutic agent-carrying stylet is provided for treating at least a portion of an indwelling catheter by locating the stylet within a portion of the catheter lumen and/or a connection means of the catheter. The therapeutic agent-carrying stylet can provide a localized therapeutic effect by administering one or more therapeutic agents directly to one or more selected features, including the luminal surfaces of the catheter, catheter openings, the connection means on the catheter, and luminal body fluids in contact with or in proximity to the catheter and/or stylet. Through repeated removal and replacement of the therapeutic stylet from the catheter, a renewable therapeutic catheter treatment method can be safely provided to the patient while the catheter remains implanted in a patient, and without the hazards of a liquid lock solution impacting the patient's systemic circulation, pulmonary system or heart. Therapeutic agent doses can also be tailored to each individual's clinical condition, and/or be tailored to the specific length, region or location along the catheter lumen.

Depending upon a patient's tested blood chemistry before, during and/or at the conclusion of their dialysis treatment, a physician or healthcare worker can make a therapeutic treatment decision for which medication and at what dose can be applied with a therapeutic treatment stylet. In this manner, insertion and catheter treatment with the medicated stylet can begin immediately following removal of the dialysis treatment connection.

By locating the therapeutic agents on a non-systemic medicated stylet, conventional, dangerous and operator-dependent liquid catheter lock chemical solutions can also be avoided, or be made more clinically effective. Therefore, in addition to avoiding the danger inherent with all liquid catheter lock solutions, a professionally trained healthcare worker may not be required for removal of the stylet before use of the catheter for fluid flow treatments or installation of a medicated stylet for improved patient care.

In an illustrative embodiment, the present invention provides a catheter treatment device having a stylet that is at least partially located in a lumen of the catheter. A therapeutic agent is located on the stylet and configured to be delivered to the catheter, such as by eluting from the surface or component of the stylet.

In another illustrative embodiment of the invention, a vascular access system is provided. The vascular access system includes a catheter having a lumen and a stylet that is at least partially located in the lumen. A therapeutic agent is located on a portion of, and configured to be administered from a component of the stylet.

In another embodiment of the invention, a method for treating a catheter is provided, which comprises the steps of inserting a stylet having a therapeutic agent into the catheter and treating the catheter by releasing the therapeutic agent.

According to still another embodiment of the invention, a catheter treatment device is provided, which comprises a stylet configured to be inserted within a lumen of a catheter and an attachment device for retaining the stylet within the lumen of the catheter. A therapeutic agent is located on at least a portion of the stylet.

### BRIEF DESCRIPTION OF THE DRAWINGS

The invention will be apparent from the description herein and the accompanying drawings, in which like reference characters refer to the same parts throughout the different views.

FIG. 1 illustrates a catheter treatment device according to an illustrative embodiment of the invention;

FIG. 2 is a side, cut-away view of a partially assembled vascular access system according to an illustrative embodiment of the invention;

FIG. 3 is a side, cut-away view of the fully assembled vascular access system of FIG. 2;

FIG. 4 is a cross-sectional view of a vascular access system having a coaxial stylet according to an illustrative embodiment of the invention;

FIG. 5 is a cross-sectional view of a vascular access system having a stylet with a rectangular cross-section, according to an illustrative embodiment of the invention;

FIG. 6 is a side, cut-away view of a portion of a vascular access system having a stylet with a helical shape, according to an illustrative embodiment of the invention;

FIG. 7 is a side, cut-away view of a portion of a vascular access system having a stylet with a balloon, according to an illustrative embodiment of the invention;

FIG. 8A is a side, cut-away view of a portion of a vascular access system having a multi-lumen catheter and multiple stylets, according to an illustrative embodiment of the invention;

FIG. 8B is a cross-sectional view of the vascular access system of FIG. 8A;

FIG. 9 is vascular access system having a protective sheath, according to an illustrative embodiment of the invention; and

FIG. 10 depicts a method according to a further embodiment of the invention.

### DETAILED DESCRIPTION

An illustrative embodiment of the present invention relates to a device for treating an indwelling catheter by locating a therapeutic agent-carrying stylet in at least a portion of the lumen of the catheter and/or within a catheter connection means. The stylet carries a therapeutic agent for treating one or more selected portions of the catheter and/or luminal body fluids in contact with or in proximity to the catheter and/or stylet. The present invention will be described below relative to an illustrative embodiment. Those skilled in the art will appreciate that the present invention may be implemented in a number of different applications and embodiments and is not specifically limited in its application to the particular embodiments depicted herein.

As used herein, the term “stylet” refers to a shaped object configured to be coupled to a catheter and at least a portion of which is configured to be inserted into at least a portion catheter lumen and/or a catheter connection means used for clinical connection of an indwelling catheter to or other interventional medical device.

The term “therapeutic agent” is intended to refer to any material capable of causing a biological or chemical effect to cells, body fluid, catheter fluids and/or catheter surfaces. The term “therapeutic agent” includes a wide variety of therapeutic agents, medications, drugs and biologically enhancing agents or chemicals capable of providing a therapeutic benefit to the catheter, luminal catheter fluids, and/or local tissue in fluid contact with the indwelling catheter. A therapeutic delivery coating or material is any bio-erodible surface or biocompatible film or coating, or drug immobilizing material which carries, elutes and/or releases a controlled amount of therapeutic agent over a prescribed period of time. The therapeutic agent can be in the form of a therapeutic delivery coating on a surface of the stylet or any suitable form known in the art.

The present invention can minimize or eliminate the dangers inherent with powerful liquid catheter lock chemical fluids by using the therapeutic agent-carrying stylet to controllably release and administer therapeutically effective doses of medication or chemical agents to targeted locations along the surface of or within a portion of an indwelling catheter. By repetitive use, removal and replacement of the medicated stylet, the present invention can provide the first known renewable and controllable therapeutic dose means to the internal areas of a catheter while the catheter remains functionally implanted in the patient. Medication doses can also be tailored along the length of the catheter and/or be made to function or release over a predetermined period of time. A timed release method for medication deployment can further be implemented for extended or pattern drug release over time. Limitations of conventional attempts to provide long-term therapeutic agent effectiveness via catheter coatings can be overcome by a repeatable and replaceable device that does not require a professionally trained healthcare worker for installation, medication dose control or medication removal due to the controlled release of the medication, without risk of accidental overdose or displacement into the patient's systemic circulation.

In an illustrative embodiment, the therapeutic agent-carrying stylet is a temporary insert, which is placed within a portion of an indwelling catheter when the catheter is not being used or performing a primary clinical function or purpose. Alternatively, the therapeutic agent-carrying stylet can be made to be used during clinical catheter use. The therapeutic agent-carrying stylet can deliver a fresh supply of a therapeutic agent, such as medication, to a selected portion of the catheter, or be made to deliver medication to all internal portions of the catheter device, as well as the external catheter connection means. Optionally, the stylet may protrude into or through either a distal catheter opening and/or a side hole of the catheter, providing a therapeutic agent to an external catheter tip, an adjacent perimeter hole surface, a side wall surface of the catheter and/or local tissue location in close proximity to the catheter or stylet.

By the use of the invention, the need for accurate fluid volume calculation, preparation, medication dosing, liquid volume installation and removal of fluids to avoid harm to the patient's systemic circulation is minimized and/or completely eliminated. Rather than injecting a liquid catheter lock solution to treat the catheter, the medicated stylet may be treated with a wide variety of clinically effective chemical agents, anti-coagulants, antiseptics, antibiotics and/or other therapeutic agents. The therapeutic agent can be made available through a variety of different release rates and doses. Among other advantageous effects, these therapeutic agents can inhibit the formation of bacterial growth within the catheter, and cellular or fibrin based proliferation that can lead to catheter occlusion of a catheter opening, catheter surface or luminal connection means over some extended period of time while the indwelling catheter is implanted in the patient.

Optionally, the stylet may be used with a safe injectable liquid catheter solution to further enhance the medicated stylet's drug release function and/or release profile from the stylet. In such an application, the liquid catheter solution may optionally be made of an inert biocompatible and non-toxic solution, such as saline, water and combinations thereof, or may be formed of a liquid gel formed with a second complimentary or reduced-strength therapeutic agent, which may further reduce the risk of using such liquid catheter lock solutions. Another example of a liquid catheter solution for use with the therapeutic agent-carrying stylet includes components of the patient's own blood. The present invention can place a specific quantity of medication, adequate to perform a therapeutic benefit in and/or around the implanted catheter, thereby minimizing or avoiding adverse systemic effect to the patient, with or without a complementing liquid catheter solution for use in combination with the patient's body fluids.

The illustrative embodiment of the present invention can also optionally provide a temporary full or partial mechanical catheter lumen occlusion means, to further prevent blood clots or fibrin sheath formation from occurring within a portion of the lumen of the indwelling catheter when the catheter is not in use. Therefore, a therapeutic agent-carrying stylet placed within an indwelling catheter can serve as both a temporary full or partial mechanical occluder to displace a certain amount of luminal area or space, and also serve as a therapeutic agent delivery device to deliver a therapeutic agent to at least a portion of an indwelling catheter or body part to therapeutically treat and or reduce biologically formed constrictions, occlusions or infections that would otherwise form within the catheter, causing an undesirable patient complications when the catheter is not in use or between medical treatment use. It is further understood that if the stylet is sized to not fully occlude a portion of the catheter lumen, it may be desirable for the operator to maintain full catheter function and or fluid flow through the catheter while the stylet remains installed in the patient's catheter.

Because the stylet provides an easily replaceable means for supplying fresh therapeutic agents when placed within the indwelling catheter, the present invention can provide a simplified means to promote catheter lumen or side hole patency, including clot-free operation and/or biofilm and/or infection prevention, even in remote locations within the body. The therapeutic agent-carrying stylet can also be made part of an external seal mechanism to keep the catheter lumen leak-proof and closed to ambient atmosphere when not in use, by mounting to an end of the stylet, a leak-proof closure or catheter tubing connection means with one end of the indwelling catheter.

The present invention is applicable for use with a catheter for a wide variety of internal organs, organ spaces, remote tissue locations, body cavities and therapeutic agent treatments. By way of example in describing the invention, a blood vessel for purposes of dialysis treatment is discussed herein. Other examples of applications of the invention include treatment of a stent, port access device, needle connection system, or bypass graft by the use of a catheter. Those in the art will appreciate that the present invention may be used with all internal organs and body cavities where a catheter may be placed, and the invention is not limited to applications for use with those organs in fluid communication with blood vessels or dialysis treatments.

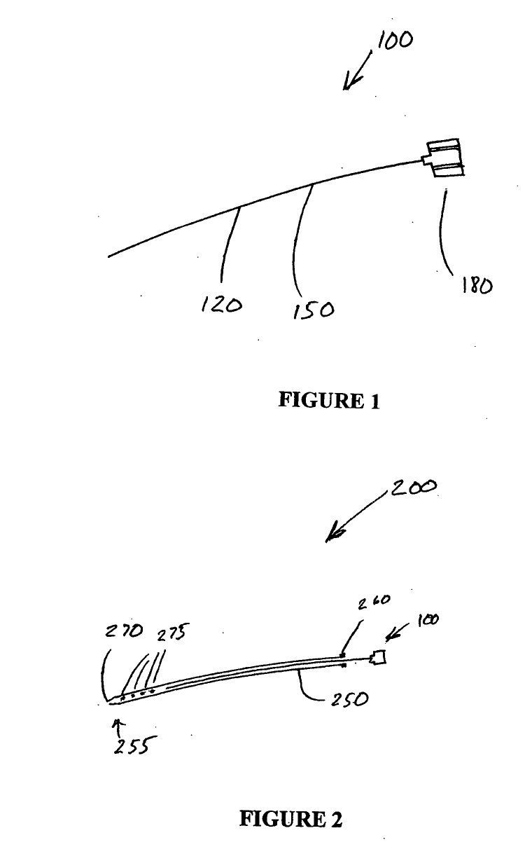

In the illustrative embodiment of the invention, a catheter treatment device 100 is provided. As illustrated by way of example in FIG. 1, the device 100 includes a stylet 150 with one or more therapeutic agents 120 located on at least a portion of the stylet 150. As used herein, the phrase “on the stylet” is intended to include locations on at least a portion of the stylet surface or within at least a portion of the structure of the stylet 150. The therapeutic agent 120 may provide a therapeutic effect to one or more components or surfaces, such as, but not limited to: a luminal surface of the catheter, luminal body fluids, body fluids in the vicinity of the catheter, tissue in the vicinity of the catheter, the connection means of the catheter, and openings of the catheter. Examples of openings in the catheter include, end holes formed in the catheter proximal and/or distal ends and side holes formed in a side wall of the catheter. For example, the therapeutic agent may be used for inhibiting undesired fibrin sheath formation or cellular proliferation of smooth muscle cells within the catheter lumen, catheter openings, and/or catheter connection means. The therapeutic agent may be transferred from the stylet 150 to a treated component or surface through direct contact or fluid contact with the component or surface.

Optionally, an attachment device 180 is mounted to an end of the stylet 150 to enable the stylet 150 to be coupled to a catheter. The attachment device preferably holds the stylet in a selected position within the catheter lumen for a selected period of time to enable treatment using the therapeutic agent. According to the illustrative embodiment, the attachment device 180 is a Leur lock cap. The attachment device 180 can comprise any suitable device suitable for coupling the stylet to an associated catheter and/or at least temporarily fixing the position of the stylet 150 relative to the catheter. For example, the stylet 150 may alternatively be coupled to the catheter via a friction fit, an internal or external threaded connection, an interference fit or any suitable means known in the art for coupling a first component to a second component.

The attachment device 180 may be configured to provide an environmental seal to an end of the catheter by forming a seal by mating with a mounting portion of the catheter. In this manner, the stylet 150 can prevent leaks from the catheter while administering the therapeutic agent within the catheter.

Table # 1 includes illustrative classes and examples of therapeutic agents that may be used within the scope of the invention. It is understood that the invention is not limited to the classes or examples of therapeutic agents in Table # 1 and may include other therapeutic agents capable of providing a localized therapeutic effect in or near the lumen of the catheter, on the surfaces of the catheter, or in the fluid in contact with the catheter and stylet. The stylet can include a combination of therapeutic agents to provide a variety of desired effects.

TABLE 1

| CLASS | EXAMPLES |
|---|---|
| Antioxidants | Alpha-tocopherol, lazaroid, probucol, phenolic antioxidant, |
| | resveretrol, AGI-1067, vitamin E |
| Antihypertensive Agents | Diltiazem, nifedipine, verapamil |
| Anti-inflammatory Agents | Glucocorticoids, NSAIDS, ibuprofen, acetaminophen, |
| | hydrocortizone acetate, hydrocortizone sodium phosphate, |
| | methylprednisolone, pemicrolimus |
| Growth Factor | Angiopeptin, trapidil, suramin |
| Antagonists | |
| Anti-platelet Agents | Aspirin, dipyridamole, ticlopidine, clopidogrel, GP IIb/IIIa |
| | inhibitors, abcximab |
| Anti-coagulant Agents | Bivalirudin, heparin (low molecular weight and |
| | unfractionated), wafarin, hirudin, enoxaparin, citrate, |
| | Tissue plasminogen activator (TPA) |
| Thrombolytic Agents | Alteplase, reteplase, streptase, urokinase, TPA, citrate |
| Bacteriacide | Selenium |
| Therapeutic agents to | Fluvastatin, colestipol, lovastatin, atorvastatin, amlopidine |
| Alter Lipid Metabolism | |
| (e.g. statins) | |
| ACE Inhibitors | Elanapril, fosinopril, cilazapril |
| Anti-hypertensive Agents | Prazosin, doxazosin |
| Anti-proliferatives and | Cyclosporine, cochicine, mitomycin C, sirolimus |
| Anti-neoplastics | microphenonol acid, rapamycin, everolimus, tacrolimus, |
| | paclitaxel, estradiol, dexamethasone, methylprednisolone, |
| | methatrexate, cilastozol, prednisone, cyclosporine, |
| | doxorubicin, ranpirnas, troglitzon, valsarten, pemirolast, |
| | pemicrolimus, SAR, protein kinase inhibitors, |
| | pemicrolimus, SAR 943 |
| Tissue growth stimulants | Bone morphogeneic protein, fibroblast growth factor |
| Gasses | Nitric oxide, super oxygenated O2 |
| Promotion of hollow | Alcohol, surgical sealant polymers, polyvinyl particles, |
| organ occlusion or | 2-octyl cyanoacrylate, hydrogels, collagen, liposomes |
| thrombosis | |
| Functional Protein/Factor | Insulin, human growth hormone, estrogen, nitric oxide |
| delivery | |
| Second messenger | Protein kinase inhibitors |
| targeting | |
| Angiogenic | Angiopoetin, VEGF |
| Anti-Angiogenic | Endostatin |
| Inhibitation of Protein | Halofuginone |
| Synthesis | |
| Anti-infective Agents | Penicillin, gentamycin, adriamycin, cefazolin, amikacin, |
| | ceftazidime, tobramycin, levofloxacin, silver, copper, |
| | hydroxyapatite, vancomycin, ciprofloxacin, rifampin, |
| | mupirocin, RIP, kanamycin, brominated furonone, algae |
| | byproducts, bacitracin, oxacillin, nafcillin, floxacillin, |
| | clindamycin, cephradin, neomycin, methicillin, |
| | oxytetracycline hydrochloride, selenium, cationic |
| | antimicrobial peptides such as MBI-226, minocylcine, |
| | pemicrolimus |
| Gene Delivery | Genes for nitric oxide synthase, human growth hormone, |
| | antisense oligonucleotides |
| Local Tissue perfusion | Alcohol, H2O, saline, fish oils, vegetable oils, liposomes |
| Nitric oxide Donative | NCX 4016 - nitric oxide donative derivative of aspirin, |
| Derivatives | SNAP |
| Gases | Nitric oxide, super oxygenated O₂ compound solutions |
| Imaging Agents | Halogenated xanthenes, diatrizoate meglumine, diatrizoate |
| | sodium |
| Anesthetic Agents | Lidocaine, benzocaine |
| Descaling Agents | Nitric acid, acetic acid, hypochiorite |
| Chemotherapeutic Agents | Cyclosporine, doxorubicin, paclitaxel, tacrolimus, |
| | sirolimus, fludarabine, ranpirnase, SAR 943, pemicrolimus |
| Tissue Absorption | Fish oil, squid oil, omega 3 fatty acids, vegetable oils, |
| Enhancers | lipophilic and hydrophilic solutions suitable for enhancing |
| | medication tissue absorption, distribution and permeation |
| Anti-Adhesion Agents | Hyalonic acid, human plasma derived surgical |
| | sealants, and agents comprised of hyaluronate and |
| | carboxymethylcellulose that are combined with |
| | dimethylaminopropyl, ehtylcarbodimide, hydrochloride, |
| | PLA, PLGA |
| Ribonucleases | Ranpimase |
| Germicides | Betadine, iodine, sliver nitrate, furan derivatives, |
| | nitrofurazone, benzalkonium chloride, benzoic acid, |
| | salicylic acid, hypochlorites, peroxides, thiosulfates, |
| | salicylanilide |

According to one aspect, the stylet 150 can be radiopaque or include one or more radiopaque portions to provide diagnostic location of the device 100 during installation, for example, under fluoroscopy. In one embodiment, the tip of the stylet 150 can be radiopaque. The use of a radiopaque stylet further provides radiopaque diagnostic location indicators for marketing an occlusion location within the catheter during installation. For example, if the stylet fails to pass through a catheter lumen area, the location of the stylet can be mapped using the radiopaque portion denote the location of the catheter occlusion under flouroscopy. Other detectable means and methods can be used when the stylet is installed using other non-invasive diagnostic techniques, ultrasound, PET scan, Cat scan, MRI, and Thermal scan and others known in the art. The stylet can also be made of non-radiopaque materials (non-metalic material) with one or more radiopaque or ultrasonic detectable coatings, or radiopaque fillers (barium sulfate/titanium oxide) or radiopaque markers commonly used for diagnostic catheter detection.

The therapeutic agents 120 may be applied to the stylet 150 through any suitable method known in the medical device drug delivery art. For example, one or more therapeutic agents may be formed as a solid film deposited onto the stylet 150. Alternatively, therapeutic agent 120 may be made as an adherent liquid that is wiped on during manufacturing or by the clinical user. A liquid ingredient can optionally be dripped onto, vapor deposited onto or liquid spray/dry solid sprayed onto the stylet. In another method, the stylet can be dipped into a liquid solution, particle suspension solution or nanoparticle dry powder of one or more therapeutic agents to apply the therapeutic agent or agents to the stylet. The medication can be made part of a coating or added as a separate manufacturing step or process. The coating can be made to elute the therapeutic agent at a selected rate. One skilled in the art will recognize that multiple coatings may be used to apply the therapeutic agent to the stylet. The medication can be either made as a soluble component in the stylet drug carrier material or added as nano-particles suspended in the drug carrier coating or material used for drug delivery on the stylet. In one embodiment, the stylet, which is preferably sterile, includes a coating that uptakes a medication, which is preferably sterile, when a therapeutic agent is applied to the device just prior to installation of the stylet into the patient's catheter by the user. Alternatively, the therapeutic agent can be applied by the manufacturer in a layer by layer lamination coating process common to the medical device industry.

The medicated stylet 150 can be made to include a reservoir, a drug-releasing pad or swab with which medications can be carried to an internal body cavity. For example, the stylet can include a full length or partial length reservoir that may be filled with a therapeutic agent. Alternatively, the stylet 150 can include a full or partial length external micro-porous swab for holding an amount of a therapeutic agent that is located over or on top of the body of the stylet. The micro-porous swab can alternatively be located partially within a hollow portion of the stylet and/or partially protruding outside of the stylet.

The catheter treatment device 100 can include a plurality of different therapeutic agent types and does to provide a variety of therapeutic effects. For example, various therapeutic agent types and doses can also be tailored along the length of the stylet to provide different therapeutic effects at different locations in, around or near the tip of the catheter, or placed near strategically located catheter holes along the radial wall surface or length of the catheter.

Alternatively or in addition, by the use of timed-release therapeutic agents and drug carrier materials and frequency of the replacement of each placed stylet offers the healthcare worker or clinician a wide variety of therapeutic treatment and dosing options to vary the therapeutic effect to an indwelling catheter.

FIG. 2 illustrates a vascular access system 200 having a catheter treatment device 100 partially inserted into a catheter 250. The mounting portion 260 of the catheter 250 is configured to mate with the attachment device 180 of the catheter treatment device 100. The catheter 250 is also illustrated with an end hole 270 at an insertion end 255, which is the end that is inserted into a patient. Side holes 275 are also shown by way of example near the insertion end 255.

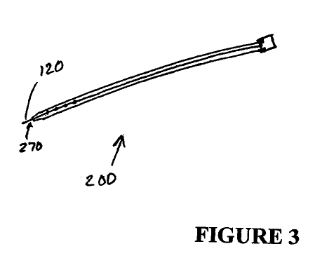

As shown in FIG. 3, the stylet 150 may also optionally be sized to extend through the end hole 270 of the catheter and thereby allow the stylet 150 to be extended through the end hole 270 and dislodge growth and/or administer therapeutic agents within the end hole 270. The extension of the catheter through the end hole may also facilitate application of the therapeutic agent to body tissue or body fluid in the vicinity of the insertion end 255 of the catheter.

One skilled in the art will recognize that the stylet 150 can have any suitable length for providing a therapeutic effect to one or more selected components of the vascular access system. For example, as shown in FIG. 3, the stylet 150 can have a length such that the end of the stylet protrudes through an end hole of the catheter. Alternatively, the stylet 150 can have a relatively short length, so as to provide a therapeutic effect to the mounting portion 260 of the catheter without extending relatively far into the catheter lumen. The stylet 150 can also have an intermediate length.

One or more components or portions of the vascular access system 200 may be color-coded to signify different characteristics. In one embodiment, the attachment device 180 and/or the catheter, or end of the catheter, may optionally be color coded to signify different characteristics. For example, a selected color of the attachment device 180 can be used to signify a therapeutic agent configuration applied on the attached stylet or a length of the stylet. The particular color of the catheter, or an end of the catheter, could signify the length of the catheter. By matching colors of the attachment device 180 and the catheter to each other, an operator can be assured of the proper length of the stylet for the implanted catheter. Optionally, a different color or multiple colors could be used to designate a stylet that is long enough to extend through or be located near the distal catheter hole located at one end of the catheter.

The stylet may be formed of one or more materials to provide an axial rigidity sufficient to enable insertion into and along the length of a catheter device without kinking the catheter or stylet. The stylet may be formed of a wide variety of materials, including shape memory metal and/or plastic. One specific example is a stainless steel wire that is flexible but has longitudinal axial strength to enable forward motion of the stylet without kinking or buckling during insertion into and within the catheter lumen. It maybe optionally advantageous to have the stylet buckle over some portion of the catheter length to increase contact with the luminal surface of the catheter when necessary, or to increase the medicated surface area of the stylet when placed within the catheter. The stylet have a strength sufficient to penetrate and/or dislodge biologically formed blockages, such as clots and fibrin plugs and webs that can form along the inner length of the catheter lumen, and/or form within a catheter side wall hole, catheter connection means, slit or opening, or end hole 270 or at other locations anywhere along the internal length of the lumen of the catheter.

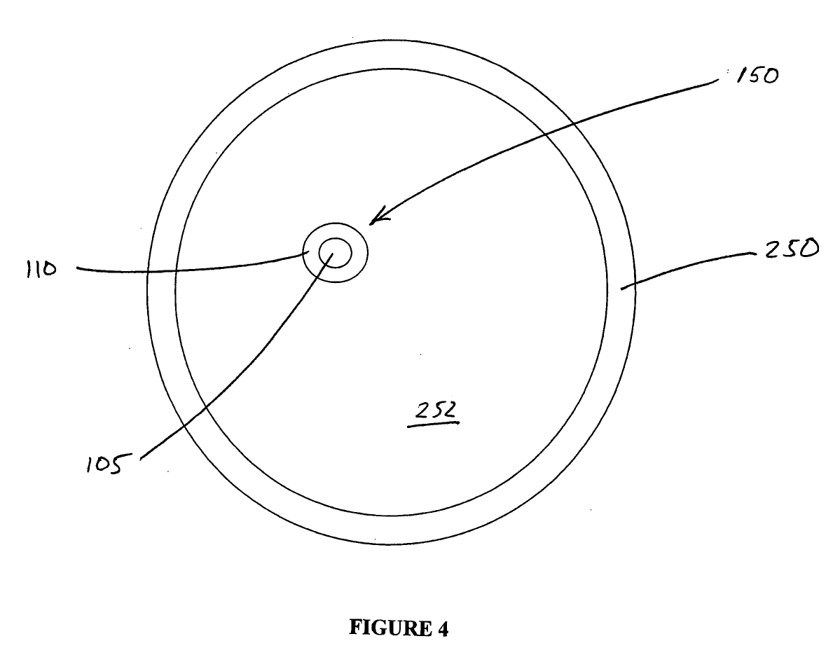

The stylet 150 can have any suitable size, shape and configuration. For example, the stylet 150 can be monolithic, multilayered or formed as a composite solid or hollow structure, or have selected portions that are either solid or hollow. The stylet can further be made of several individual elements at equal or different lengths that when bundled together in at least one location, whereby such multi-element components act and perform as one stylet unit for installation within a catheter. In one example, shown by way of example in FIG. 4, the stylet 150 can be a coaxial structure having a core 105 and an outer layer 110. In one implementation, the core 105 is formed of stainless steel and the outer layer 110 is formed of plastic. The stylet can be made from a wide variety of smooth surface finishes, rough surface finishes and combinations thereof. The surface of the stylet can be hard or soft, porous or non-porous, slippery or non-slippery and combinations thereof. Examples of materials include, but are not limited to, 316 L stainless steel, cobalt chromium metal alloy, TEFLON, nylon and PET plastics, including shape memory materials, such as nitinol, in plastic and alloy composites.

The stylet can also have a surface or coating that is hydrophilic or hydrophobic or a combination of hydrophilic and hydrophobic materials. For example, a coating or coatings on the stylet including the therapeutic agent can be formed of a material that is hydrophilic or hydrophobic or a combination of hydrophilic and hydrophobic materials.

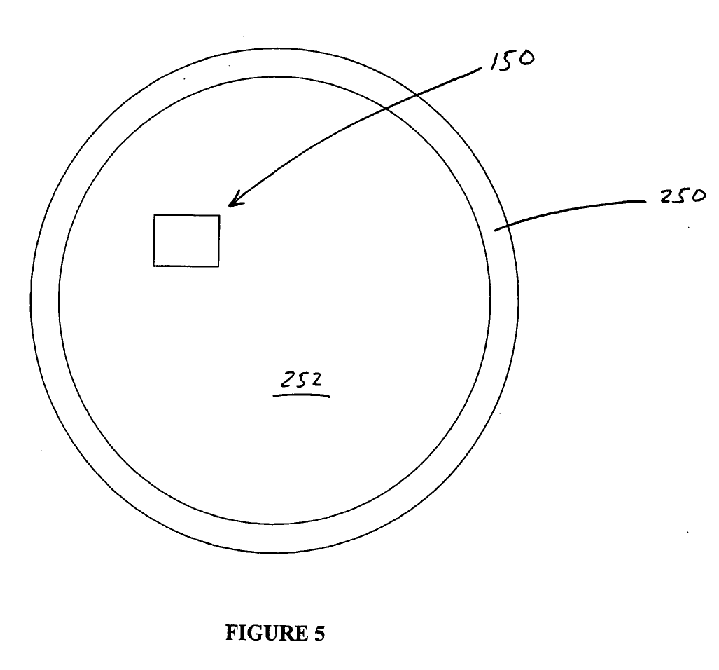

A variety of stylet 150 cross sections are within the scope of the invention. Examples include circular, oval, annular, square, rectangular, flat, cross-shaped, crescent-shaped and other suitable shapes, including several combinations of different cross-sectional shapes, edged finish and lengths, as shown in FIGS. 4 and 5. The cross-section of the stylet 150 can be uniform in the size and shape along the length of the stylet, or the cross-section can vary in size and/or shape.

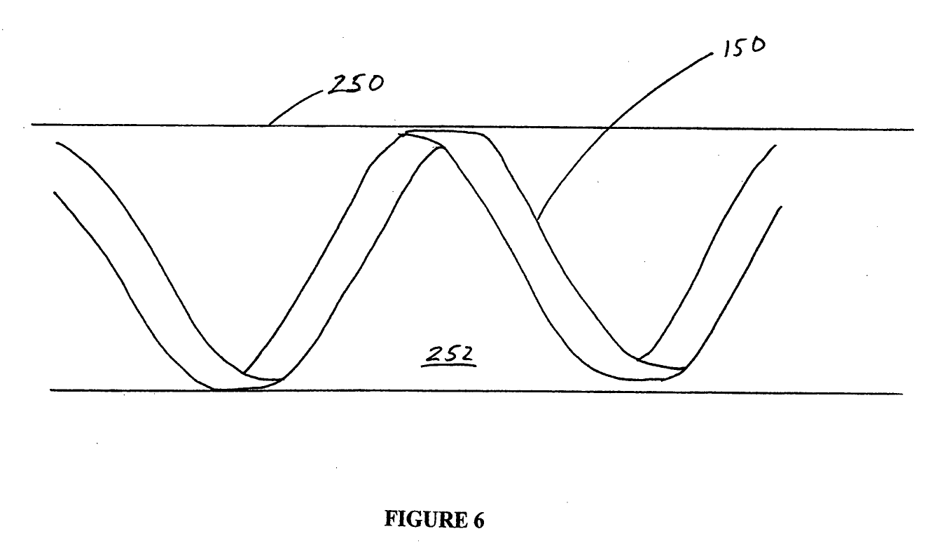

The shape of the stylet 150 is not limited to a particular configuration. Examples of stylet 150 shapes along the length of the stylet include, but are not limited to, linear, as shown in FIGS. 1-3, or helical, as shown in FIG. 6. While the linear stylet of FIGS. 1-3 and the helical-shaped stylet of FIG. 6 are formed of a single wire construction, one skilled in the art will recognize that the stylet can alternatively comprise multiple wires wound together. The stylet can alternatively have a curved shape, a zigzagged shape, a spiral shape other any other suitable shape along a lengthwise direction.

The stylet 150 can be shaped using any suitable means known in the art, including, but not limited to molding and extrusion.

By the use of a small diameter stylet 150, a catheter clamp may be used along an outside surface of the catheter to prevent flow of fluid through the catheter, without requiring removal of the stylet. The catheter clamp may later be released without requiring adjustment to the stylet. If the stylet is made of a resilient material, no change in shape to the stylet may result from the repeated use of catheter clamps.

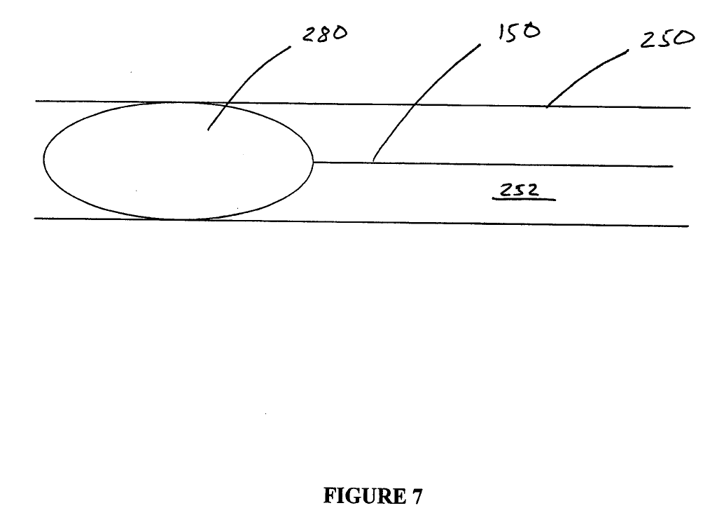

The stylet may be sized to be used for temporary full or partial occlusion of the catheter 250, or be made to be minimally occlusive to the catheter and its openings, and full or partially occluding an adjacent catheter side wall hole, channel or distal or proximal end hole. In one embodiment, the stylet 150 may be sized to occlude the lumen 252 of an associated catheter by having an outer diameter, along at least a portion of the stylet 150, that is substantially similar to the diameter of the lumen 252. The stylet may be designed to fully occlude or partially occlude the distal or proximal end hole of the catheter. The stylet may partially obscure, interfere with or partially block one or more catheter side holes along the length of the catheter lumen. In another embodiment, the stylet 150 may include a fully occluding or partially occluding inflatable balloon at some location along the stylet length. The inflatable balloon 280 may be provided on the stylet 150, as shown by way of example in FIG. 7. The balloon may be installed into the catheter device in an un-inflated condition during insertion of the stylet 150 and at least partially inflated while the stylet 150 is located within the catheter 250. If the balloon 280 located on the stylet is sufficiently inflated so as to at least partially occlude some portion of the catheter lumen 252, inflation of the balloon may physically dislodge or compress some portion of an undesirable biologically formed growth or catheter lumen obstruction located along the sides of the catheter lumen 252.

The location of the stylet 150 within the lumen 252 of the catheter 250 may vary. The stylet 150 may be located in the center of the lumen 252, or it may be located off center. The location of the stylet 150 may vary along the length of the catheter 250. For example, the stylet 150 may be located at the center of the lumen 252 at the attachment device 180 and along the side wall of the catheter lumen 252 at locations where the catheter 250 is bent, forcing at least a portion of the stylet to make direct contact with the inside surface of the indwelling catheter.

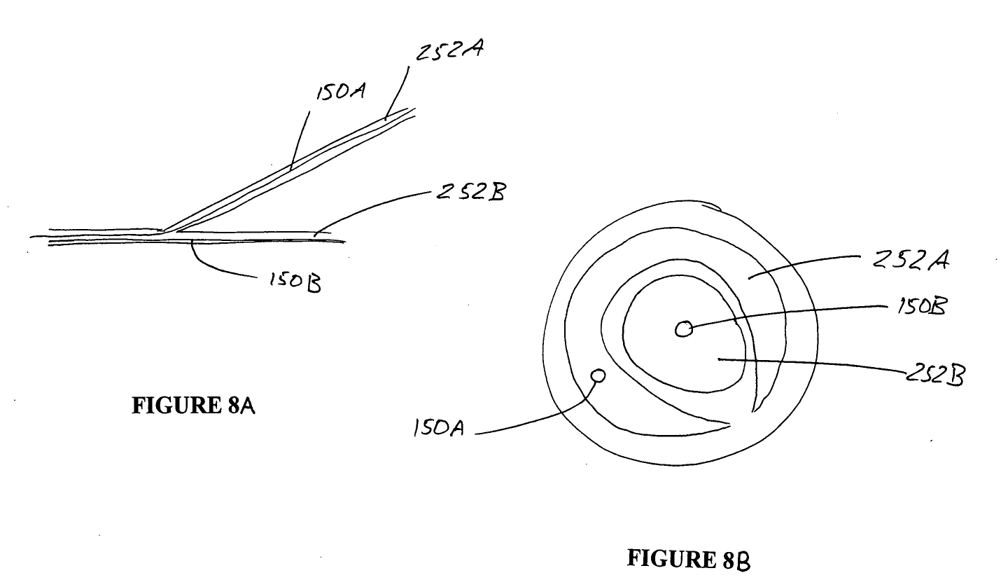

In one embodiment, multiple stylets can be used to provide a therapeutic agent. For example, as shown in FIGS. 8A and 8B, a vascular access system can include multiple stylets 150A, 150B, with one in each lumen 252A, 252B of a multi-lumen catheter. In another embodiment of the invention, multiple stylets can be simultaneously used in a single lumen of a catheter. If an attachment device 180 is used, multiple stylets may optionally be attached to a single attachment device 180 for multiple stylet placement into a single lumen catheter or for multiple stylet placement into multiple lumens of a multi-lumen catheter.

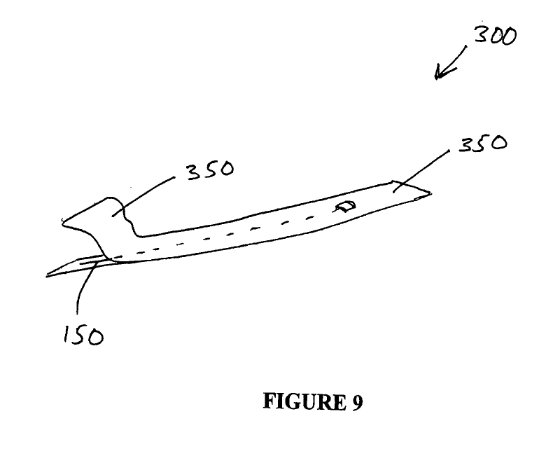

According to a further illustrative embodiment, a catheter treatment device 300 may be provided with a collapsible, partially collapsible or non-collapsible external protective sheath 350 to minimize direct handling of a sterile stylet and to prevent contact of the stylet with non-sterile surfaces. Use of an external protective stylet sheath or cover can further aid in the proper sterile technique handling of the catheter and stylet during insertion of a sterile stylet 150 into the sterile lumen of a catheter 250. An example of a protective sheath 350 according to an illustrative embodiment of the invention is shown in FIG. 9. The protective sheath 350 is shown having an open end to allow insertion of an end of the stylet 150 into a catheter. The protective sheath 350 may be formed of a wide variety of materials capable of providing a flexible barrier to maintain a sterile environment, such as, for example, polyethylene, polypropylene or blended PET film material. In an illustrative example, two layers of polyethylene film can be mounted to each other along their perimeters, forming a pocket in which the catheter treatment device 300 is located. Upon use, the polyethylene layers can be separated at least at one end, exposing a portion of the catheter treatment device 300, while allowing the operator to install the stylet into the catheter without direct contact of the sterile stylet during handling and catheter insertion. The catheter treatment device 300 can then be inserted into a catheter and the protective sheath 350 may be removed and discarded.

In a similar embodiment, a barrier film bag or transfer sleeve can serve the purpose of a protective containment means during stylet catheter installation. The protective containment means can remain affixed to the catheter stylet connection during a portion of the time or for the entire duration of which the stylet remains installed within the catheter. The protective containment means can then allow the stylet to be extracted out of the catheter within the protective containment sleeve, without direct contact by the operator during the removal of the stylet from the catheter. The protective containment means can further provide continued protection and containment of the extracted stylet during handling by the operator for ultimate disposal or for laboratory testing, including bacterial colonization testing, fluid and cell growth sampling.

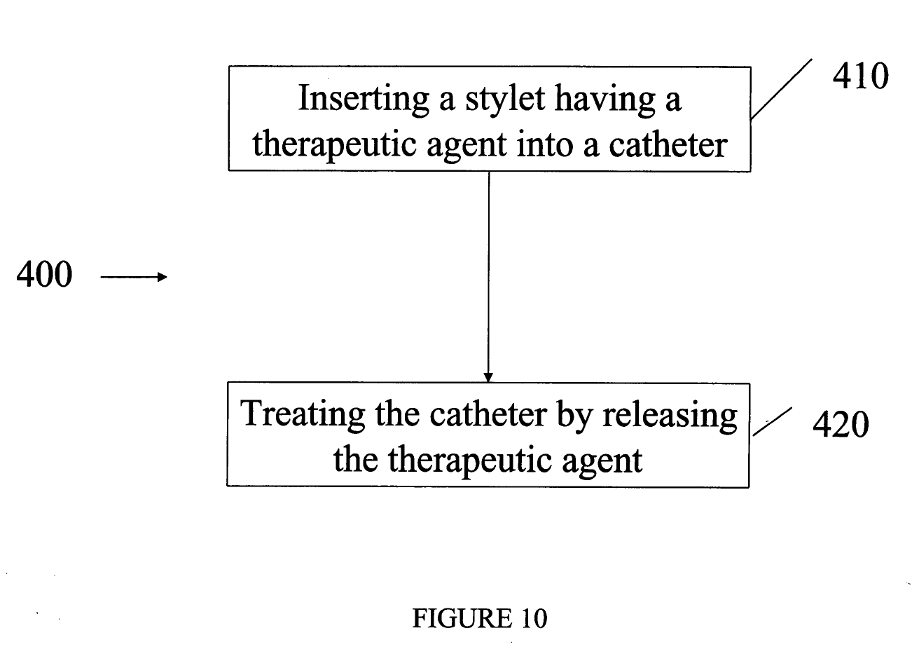

According to a further embodiment of the invention, a method 400 of treating a catheter is provided. As illustrated in FIG. 10, the method 400 includes the step 410 of inserting a stylet having a therapeutic agent into the catheter. The catheter is then treated, step 420, by controlled release of the therapeutic or bioactive agents delivered by the installed medicated stylet. The medicated stylet treatment zone may be administered to the lumen of the catheter and/or to a targeted portion of the catheter lumen, such as an end hole or side hole, luminal reservoir or side wall opening and cavity of the catheter.

The present invention has been described by way of example, and modifications and variations of the described embodiments will suggest themselves to skilled artisans in this field without departing from the spirit of the invention. Aspects and characteristics of the above-described embodiments may be used in combination. The described embodiments are merely illustrative and should not be considered restrictive in any way. The scope of the invention is to be measured by the appended claims, rather than the preceding description, and all variations and equivalents that fall within the range of the claims are intended to be embraced therein.

What is claimed is:

1. A catheter treatment device, comprising:
a stylet, at least partially located in a lumen of said catheter and coupled to the catheter, and
a therapeutic agent, located on at least a portion of said stylet for delivery to the catheter.

2. The device of claim 1, wherein said therapeutic agent is configured to treat a portion of said catheter lumen.

3. The device of claim 1, wherein the therapeutic agent is configured to treat body tissue in contact with said catheter.

4. The device of claim 1, wherein the therapeutic agent is configured to treat body tissue in fluid contact with said catheter.

5. The device of claim 1, wherein the therapeutic agent on the catheter treatment device is configured to treat fluid located in said catheter lumen that is in fluid contact with the catheter treatment device.

6. The device of claim 1, wherein said therapeutic agent is configured to treat a portion of a luminal surface of said catheter by direct contact of the catheter lumen with the catheter treatment device.

7. The device of claim 1, wherein said therapeutic agent is configured to treat an opening in the catheter via contact with the catheter treatment device.

8. The device of claim 7, wherein said therapeutic agent treats the opening through fluid contact between the catheter treatment device and the opening in the catheter.

9. The device of claim 7, wherein the opening is one of a side hole in the catheter and an end hole in the catheter.

10. The device of claim 1, wherein the therapeutic agent is configured to treat a portion of a reservoir of the catheter lumen through contact of the catheter treatment device with the reservoir.

11. The device of claim 10, wherein the catheter treatment device is in fluid contact with the reservoir.

12. The device of claim 1, wherein said therapeutic agent is configured to treat a portion of an external surface of the catheter via fluid transmission with said catheter.

13. The device of claim 1, wherein said therapeutic agent is an anti-coagulant.

14. The device of claim 1, wherein said therapeutic agent is an antiseptic.

15. The device of claim 1, wherein said therapeutic agent is an anti-infective agent.

16. The device of claim 1, wherein said therapeutic agent is a mixture including at least one of an antiseptic, an anti-infective agent, an anti-coagulant agent, an anti-inflammatory agent, an anti-proliferative agent, an anti-platelet agent, a tissue plasminogen activator and a thrombolytic agent.

17. The device of claim 1, wherein said therapeutic agent inhibits a formation of bacterial growth on at least one of the catheter, a luminal surface of the catheter and lumen fluid within the catheter.

18. The device of claim 1, wherein the therapeutic agent inhibits biofilm formation on at least one of the catheter, a luminal surface of the catheter and lumen fluid within the catheter.

19. The device of claim 1, wherein the therapeutic agent limits biofilm formation to a selected area of the catheter lumen in the vicinity of the catheter treatment device.

20. The device of claim 1, wherein the said therapeutic agent is located on at least a portion of a surface of the stylet.

21. The device of claim 1, wherein said therapeutic agent is located within a structure of the stylet.

22. The device of claim 1, wherein said therapeutic agent comprises a plurality of therapeutic agents located on said stylet.

23. The device of claim 1, wherein said stylet is formed of metal.

24. The device of claim 1, wherein the stylet is formed of a non-metallic material.

25. The device of claim 24, wherein said non-metallic material is a polymer.

26. The device of claim 1, wherein said stylet has a uniform cross-section.

27. The device of claim 1, wherein said stylet is hollow.

28. The device of claim 1, wherein said stylet is solid.

29. The device of claim 1, wherein the stylet has at least one of a round cross section, an oval cross section, a rectangular cross-section, a cross-shaped cross-section, a flat elongated cross section and a curved cross section

30. The device of claim 1, wherein the stylet includes a radiopaque portion.

31. The device of claim 1, wherein said stylet is formed from a therapeutic matrix composition with at least one coating.

32. The device of claim 31, wherein said coating includes a surface that is one of hydrophilic, a hydrophobic, and combinations thereof.

33. The device of claim 1, wherein said stylet is formed in a helical shape along a lengthwise direction.

34. The device of claim 1, wherein said stylet is formed in a spiral shape along a lengthwise direction.

35. The device of claim 1, wherein said stylet has an elongated substantially straight shape along a lengthwise direction.

36. The device of claim 1, wherein said stylet has a zigzag shape along a lengthwise direction.

37. The device of claim 1, wherein the stylet has a curved shape along a lengthwise direction.

38. The device of claim 1, wherein said stylet has a coaxial structure comprised of a first material forming a core and a second material forming an outer layer.

39. The device of claim 1, further comprising a balloon mounted to said stylet, said balloon adapted to be inflated to a larger diameter than a diameter of said stylet.

40. The device of claim 1, wherein at least a portion of said stylet has a diameter substantially similar to a diameter of said lumen.

41. The device of claim 1, further comprising a protective sheath encasing said stylet and said therapeutic agent.

42. The device of claim 41, wherein said protective sheath is coupled to said catheter.

43. The device of claim 42, wherein the protective sheath remains attached to said catheter when the stylet is inserted into a catheter.

44. The device of claim 1, wherein the stylet comprises a single wire.

45. The device of claim 1, wherein the stylet comprises a plurality of wires wound together.

46. The device of claim 1, wherein said stylet further comprises an attachment device for coupling the stylet to the catheter.

47. The device of claim 46, wherein the attachment device is located at a first end of the stylet for coupling the stylet to a first end of the catheter.

48. The device of claim 46, wherein the attachment device is configured to seal the catheter.

49. A vascular access system, comprising:
a catheter having a lumen;
a stylet, at least partially located in said lumen; and
a therapeutic agent located on said stylet for delivery to the catheter.

50. The system of claim 49, wherein said catheter has a first end adapted for implanting in a patient and a second end forming a connection means for coupling said catheter to a dialysis machine.

51. The system of claim 49, wherein said stylet protrudes through a hole in said first end of said catheter.

52. The system of claim 49, wherein said stylet does not protrude through a hole in said first end of said catheter.

53. The system of claim 49, wherein the stylet is coupled to said second end of said catheter.

54. The system of claim 49, further comprising a Leur lock cap mounted to an end of said stylet.

55. The system of claim 54, wherein the stylet is coupled to said catheter by the Leur lock cap being coupled to an end of the catheter.

56. The system of claim 49, wherein the stylet is sized to occlude said lumen.

57. The system of claim 49, wherein the stylet is sized to not occlude said lumen.

58. The system of claim 49, further comprising a plurality of stylets, each of said stylets adapted to be inserted in at least one of a plurality of said catheters, each of said catheters having said lumen.

59. The system of claim 49, wherein said catheter is a multi-lumen catheter.

60. The system of claim 49, wherein said therapeutic agent is configured to treat said lumen.

61. The system of claim 49, wherein said therapeutic agent is configured to treat an end hole of said catheter.

62. The system of claim 49, wherein said therapeutic agent is configured to treat an external surface of the catheter.

63. The system of claim 49, wherein said therapeutic agent is least one of an antiseptic, an anti-infective agent, an anti-coagulant agent, an anti-inflammatory agent, an anti-proliferative agent, an anti-platelet agent, a tissue plasminogen activator and a thrombolytic agent.

64. The system of claim 49, wherein said therapeutic agent is an antiseptic.

65. The system of claim 49, wherein said therapeutic agent is an antibiotic.

66. The system of claim 49, wherein said therapeutic agent inhibits a formation of bacterial growth within the catheter.

67. The system of claim 49, wherein said therapeutic agent is located on within a structure of the stylet.

68. The system of claim 49, wherein said therapeutic agent comprises a plurality of therapeutic agents located on said stylet.

69. The system of claim 49, wherein said stylet has a coaxial structure comprised of a first material forming a core and a second material forming an outer layer.

70. The system of claim 49, further comprising a balloon mounted to said stylet, said balloon adapted to be inflated to a larger diameter than a diameter of said stylet.

71. The system of claim 49, wherein at least a portion of said stylet has a diameter substantially similar to a diameter of said lumen.

72. The system of claim 49, further comprising a protective sheath encasing said stylet and said therapeutic agent, said protective sheath adapted to be removed from said stylet and said therapeutic agent.

73. The system of claim 49, further comprising a protective sheath encasing said stylet, said therapeutic agent and said catheter, said protective sheath adapted to be removed from said stylet, said therapeutic agent and said catheter, when the catheter is inserted into a patient.

74. A method for treating a catheter, comprising the steps of:
inserting a stylet having a therapeutic agent into said catheter; and
treating said catheter by releasing said therapeutic agent.

75. The method of claim 74, wherein said treating step administers said therapeutic agent to a lumen of said catheter.

76. The method of claim 75, wherein said therapeutic agent is least one of an antiseptic, an anti-infective agent, an anti-coagulant agent, an anti-inflammatory agent, an anti-proliferative agent, an anti-platelet agent, a tissue plasminogen activator and a thrombolytic agent.

77. The method of claim 75, wherein said therapeutic agent is an antiseptic.

78. The method of claim 75, wherein said therapeutic agent is an antibiotic.

79. The method of claim 74, wherein said treating step administers said therapeutic agent to an end hole of said catheter.

80. A catheter treatment device, comprising:
a stylet configured to be inserted within a lumen of a catheter;
an attachment device for retaining the stylet within the lumen of the catheter, and
a therapeutic agent, located on at least a portion of said stylet.

81. The catheter treatment device of claim 80, wherein said attachment device is located on a first end of said stylet for coupling the stylet to a first end of the catheter.

82. The catheter treatment device of claim 81, wherein the attachment device seals said first end of the catheter.

83. The catheter treatment device of claim 80, wherein said attachment device is configured to couple to a connection means on the catheter for connecting the catheter to a dialysis machine.

84. The catheter treatment device of claim 80, wherein said attachment device comprises a Leur lock cap configured to couple to a connection on a first end of the catheter.

85. The catheter treatment device of claim 80, wherein said attachment device retains the stylet within the lumen via friction fit.
